(19) United States
(12) Patent Application Publication
(10) Pub. No.: US 2016/0287566 A1
(43) Pub. Date: Oct. 6, 2016

(54) Anti-demodectic active agents and topical compositions for the treatment of demodicosis in humans and animals

(71) Applicant: Thomas Lynn Busby, Phoenix, AZ (US)

(72) Inventor: Thomas Lynn Busby, Phoenix, AZ (US)

(21) Appl. No.: 14/678,940

(22) Filed: Apr. 4, 2015

(51) Int. Cl. A61K 31/4412; A61K 47/10; A61K 47/44; A61K 31/4164; A61K 9/00; A61K 47/14

(52) U.S. Cl. CPC A61K 31/4412; A61K 9/0014; A61K 47/14; A61K 47/44; A61K 31/4164; A61K 47/10

(57) ABSTRACT

The present invention is directed to topical compositions and methods for preventing and treating demodicosis and the symptoms of demodectic mites, including blepharitis, meibomian gland dysfunction, type 1 flushing rosacea, type 2 acne rosacea, type 4 ocular rosacea, demodicosis gravis, granulomatous rosacea, and pityriasis folliculorum, by topical administration of a therapeutically effective amount of piroctone olamine, and in some embodiments, climbazole.

### BACKGROUND OF THE INVENTION

Demodex is a commensal ecto-parasitic mite that belongs to family Demodicidae, class Arachnida, and order Acarina. All cutaneous diseases caused by Demodex mites are subsumed under the terms demodicosis. Most people and most mammals are merely carriers of Demodex mites and do not exhibit signs or symptoms. Demodicosis is therefore considered the result of internal host-factors, and most likely an innate immune response.

The inflammatory results of demodicosis are severe, and can disfigure the skin and cause the eyes to dry out. Quality of life suffers greatly from blood-shot eyes, constant crusting of the eyelids and eyelashes, burning, itching and foreign-body sensation in the eyes, and a decrease in contrast sensitivity and the quality of vision. Quality of life also suffers greatly from living day-to-day with red skin and volcano-like papules on the face, a significant, disfiguring facial disorder.

In humans, numerous medical studies report elevated densities of Demodex cause demodicosis, such as rosacea and blepharitis. Current methods to treat ocular demodicosis in humans are labor intensive: once-weekly, half-hour treatments by a medical doctor, who carefully doses and scrubs the base of the eyelids with 50% tea tree oil, and then scrubs off the tea tree oil and the dead mites with a mild shampoo. Doctor office treatments are supplemented by daily patient use of eyelid margin scrubbing with single use, disposable paper swabs containing 5% tea tree oil or a derivative thereof, terpinen-4-ol. Tea tree oil treatments for rosacea are not suitable for treating the whole face because leave-on self-administered treatment-concentrations cannot exceed 5% tea tree oil, due to the irritating properties of tea tree oil, but demodectic mites in vivo are not affected by tea tree oil concentrations under 20%. Moreover, tea oil and its derivatives have a high potential for skin irritation and often cause their own allergic reactions, inflaming the skin and burning the cornea.

Topical 1.0% ivermectin, sold as Soolantra, is a new, effective, self-administered, anti-demodectic treatment for rosacea. Soolantra is a spot-treatment cream applied to the face. However, ivermectin cannot be used near the eyes or eyelashes because it will burn the cornea, and thus cannot be used to treat blepharitis, meibomian gland dysfunction, or ocular rosacea. Moreover, Demodex do not reside only on the face, and may move from one part of the body to the face, such that spot treatment of Demodex is not as functional as a full-body treatment.

Demodicosis is likewise a common skin disease in non-human mammals, especially dogs and cattle. Usually referred to as demodectic mange, or sometimes red mange or follicular mange, demodicosis is an inflammatory parasitic skin disease in which the affected animal is burdened by an elevated density of Demodex mites. Demodicosis in non-human mammals is usually treated with oral ivermectin or topical ivermectin, or with Amitraz and Mitiban as dips and sprays, but horses and some types of dogs cannot tolerate these insecticide-based drugs and toxic or lethal consequences can occur to the animal-patient and to the person applying them. Demodicosis in non-human mammals can also be treated with 70% elemental sulfur, although no more than 10% elemental sulfur is recommended for use on humans because higher concentrations burn human skin, but in any concentration, sulfur will burn the cornea and emits an overwhelming smell of rotten eggs that cannot be masked.

An advantage of the present invention is that a therapeutic amount of the topical composition is non-toxic and non-irritating, so the present compositions can be applied daily and topically to the entire body of the person or animal requiring treatment, including the margins of the eyes. Full-body application of the compositions comprising the invention, including the scalp and hair, prevents Demodex from persisting on untreated parts of the body and then re-infesting the face and eyes. In addition, the present invention does not have an annoying smell, and unlike tea tree oil the present invention is non-irritating to the cornea and skin, and application of the present invention does not require the time, skill and expense of a treating physician.

Accordingly, the compositions comprising the invention are useful not only to humans suffering from demodicosis, blepharitis, and the subtypes of rosacea, but also, for example, to cattle ranchers, because demodicosis causes cattle to become underweight and the hides to become perforated and un-useable as leather. Self-administered, easy to use, full-body, non-irritating to the cornea, topical, effective, non-toxic, anti-demodectic treatments are functional and novel.

(8) SUMMARY OF THE INVENTION The present invention is directed to the prevention and treatment of the signs and symptoms of demodicosis in humans, including but not limited to blepharitis, meibomian gland dysfunction, type 1 flushing rosacea, type 2 acne rosacea, type 4 ocular rosacea, demodicosis gravis, granulomatous rosacea, and pityriasis folliculorum, by applying a therapeutically effective amount of topical compositions containing piroctone olamine. The present invention is also directed to the prevention and treatment of demodicosis in mammals, including but not limited to demodectic mange. Embodiments that include climbazole are directed to the competitive relationship between Demodex and Malassezia, because both are commensal ecto-parasites that metabolize the host's body sebum, such that reducing the numbers of one allows the numbers of the other to increase and become pathogenic to the mammalian host. Features, aspects, and advantages of the present invention will become more apparent from the following description of preferred embodiments of the invention.

### BRIEF DESCRIPTION OF THE SEVERAL VIEWS OF THE DRAWINGS

Not applicable.

### DETAILED DESCRIPTION OF THE INVENTION

The methods and compositions described herein are directed to the use of cosmetic compositions containing piroctone olamine [1-Hydroxy-4methyl-6-(2,4,4-trimethylpentyl)-2(1H) pyridinone, 2-aminoethanol salt, CAS #68890-66-4], and in certain embodiments, climbazole [1-(4-Chlorophenoxy)-1-(imidazol-1-yl)-3,3-dimethyl-2-butanone, CAS #38083-17-9], for the prevention and treatment of demodicosis, which some humans and some non-human mammals experience in the presence of excessive numbers of Demodex mites. The methods and compositions described herein are topical applications of a rinse-off shampoo/shower gel and a leave-on skin lotion.

Without intending to be bound by theory the compositions described herein containing piroctone olamine and in certain embodiments, climbazole, suppress Demodex, inhibit Demodex reproduction, kill Demodex, and prevent re-infestation, or provide a combination of any of the foregoing effects, such that the compositions are useful innovations for treating demodicosis. Use of the term “demodicosis” in the claims and specifications is defined to include demodicidosis, demodectic mite infestations, blepharitis, meibomian gland dysfunction, type 1 flushing rosacea, type 2 acne rosacea, type 4 ocular rosacea, demodicosis gravis, granulomatous rosacea, and pityriasis folliculorum in humans, and demodectic mange in non-human mammals. These signs and symptoms may be associated with other diseases and/or causes.

Blepharitis is a subtype of demodicosis and is treated by the compositions comprising the invention. Blepharitis is the general term for inflammation that affects the eyelids, eyes, and eyelashes. The main symptoms of blepharitis are itching, burning, and foreign body sensation. Signs of blepharitis include crusting of the eyelids, dry eyes (lipid tear deficiency), red eyes (conjunctival hyperemia), cylindrical dandruff on the eyelashes (sleeves or collarettes), lid margin inflammation (blepharoconjunctivitis), meibomian gland dysfunction, misdirected eyelashes (trichiasis), and loss of eyelashes (madarosis).

Each of the subtypes of rosacea is a subtype of demodicosis and is treated by the compositions comprising the invention. Signs of type 1 flushing rosacea include flushing of the face, facial redness (erythema) and broken blood vessels (telangiectasia). Signs of type 2 acne rosacea include facial papules and pustules (papulopustular rosacea), plus flushing and telangiectasia of type 1 rosacea. Signs of type 4 ocular rosacea are identical to blepharitis, but include signs of type 1 rosacea or type 2 rosacea. Granulomatous rosacea is similar to type 2 acne rosacea, but with smaller or flatter papules and sometimes without flushing and telangiectasia of type 1 rosacea. Demodicosis gravis and pityriasis folliculorum can be alternate terms for granulomatous rosacea, but with less background skin-redness. Demodicidosis is a term found in older literature, and has been supplanted by demodicosis.

Two demodectic mite species inhabit human skin. D. folliculorum, which can reside in any hair follicle (follicular infundibulum) located anywhere on the body, including the nose and ears; and, D. brevis, which is specialized to inhabit only the sebaceous glands, again, anywhere on the body, but its habitat is deeper in the skin than D. folliculorum. As a result, D. folliculorum can be brought to the surface via single hair twirling methods, and can be counted via a standardized skin surface biopsy with super glue, or by microscopy examination, but D. brevis cannot be seen or counted except by surgical removal of one or more sebaceous glands inhabited by mites.

D. folliculorum can cause blepharitis associated with disorders of the eyelashes (anterior blepharitis) and disorders appearing as granulomatous acne-like cysts, papules and pustules. D. brevis can cause posterior blepharitis and meibomian gland dysfunction. Both types of Demodex can cause crusting of the eyelids, dry eyes, red eyes, red eyelid margins, itching, foreign body sensation, light sensitivity, lash fallout, misdirected lashes, pinholes in the cornea (punctate epithelial erosions), and corneal ulceration in severe longstanding cases. Both types of Demodex can cause type 1 flushing rosacea (erythematotelangiectatic rosacea), type 2 acne rosacea (papulopustular rosacea), type 4 ocular rosacea, pityriasis folliculorum (follicular papules and plugs associated with dry scaling and diffuse facial erythema), granulomatous rosacea, and demodicosis gravis.^{1 2 3 4 5} Demodectic blepharitis and demodectic rosacea are often underdiagnosed or misdiagnosed.⁶ Demodex are commensal to all mammals and therefore finding demodectic mites is inconclusive, because only some individuals react negatively to the presence of Demodex. Therefore, a favorable response to anti-demodectic treatment is a more functional, evidence-based, diagnostic method. Each of the medical disorders, symptoms, and signs described in this paragraph are hereinafter collectively referred to as “inflammatory response to demodex.”

Demodex are arachnids, having biting mouth parts and eight stubby legs, and reproduce sexually. Both types of demodectic mites become commensal to the skin of all humans, increasing in population-density with the age of the host and infest nearly 100% of persons age 70 and older. Demodex are specialized to feed on body sebum. However, the meibomian glands, a subtype of sebaceous gland, provide the richest source of sebum and are a prime location for D. brevis. The meibomian glands are numerous and vertically oriented inside the skin of the upper and lower eyelids. Tubules exit the meibomian glands onto the surface of the cornea are immediately adjacent to the cornea, and demodectic mites obtain entry to the meibomian glands through the meibomian tubules.

All species of Demodex that affect mammals, including humans, are hereinafter referred to as “demodex” or “demodectic mites.” Male and female demodex do not reside in the same hair follicle or sebaceous gland, probably due to a poly-microbial quorum sensing mechanism.⁷ As a consequence of their cloistered pre-maturity habitat, mature male demodex crawl out onto the surface of the skin, especially at night as they are photosensitive, in order to find and mate with female demodex. When male demodex leave the shelter of the sebaceous gland or hair follicle in order to mate, they are exposed to residues on the skin of the topical compositions described in this invention.

Demodectic mites rapidly desiccate and die when removed from the body and cannot reproduce in vitro, and skin conditions triggered by demodex are part of a complex innate immune response not yet fully explained by medical research. Thus, Koch's postulates are not useful, and research is typically focused on logical observations, such as cause and effect, of humans and other mammals who exhibit symptoms considered common to demodex. Medical research, cited herein, identifies the inflammatory response to demodex as characterizing the symptoms of blepharitis, meibomian gland dysfunction, flushing rosacea, acne rosacea, and ocular rosacea.^{8 9 10} Human in vivo testing of topical compositions containing 0.14% piroctone olamine (effective treatments may range from 0.10 to 0.18%), when formulated as an oil in water emulsion as described herein, reduced and eliminated the symptoms of blepharitis, meibomian gland dysfunction, and papules about the eyelid margins and lip margins. Treatment is preferentially once-daily, at night, with topical applications, of a rinse-off shampoo/shower gel, and of a leave-on skin lotion. Treatment of the inflammatory response to demodex with compositions comprising the invention is novel and not previously reported. Treatment of the inflammatory response to demodex includes treatment of type 1 flushing rosacea, type 2 acne rosacea, type 4 ocular rosacea, demodicosis gravis, granulomatous rosacea, and pityriasis folliculorum, and treatment of demodicosis in mammalian quadrupeds, namely, demodectic mange.

Demodex are believed to have a life-span of approximately 23 days.¹¹ Using once-daily topical applications at night of the rinse-off shampoo/shower gel, containing piroctone olamine at concentrations of 0.14% by weight and climbazole at concentrations of 0.07% by weight, test subject symptom-improvement was remarkable on day-24 of treatment, with the eyelashes and the margins of the eyes exhibiting steady elimination of the symptoms typically associated with demodectic mites and blepharitis, including reduction of dry eyes, red eyes, swollen eyelids and crusty cylindrical dandruff (collarettes) around the base of the eyelashes. During days 25-60 of once-daily use at night of the rinse-off and the leave-on compositions described herein, all collarettes were removed, and crusting of fluids around the margins of the eye and erythema of the conjunctiva receded and vanished. During days 45-70, gentle pressure and gentle scraping along the margins of the eyelids revealed and expressed clogs of detritus, at first waxy and in the following days becoming watery, from the open tubules of the meibomian glands. On days 45-90 of once-daily use of the compositions described herein, papules about the eyes and lips began to recede. Sebaceous glands and meibomian glands clogged by mite-detritus atrophy somewhat, extending the time for complete meibomian gland and pilosebaceous unit healing to approximately 110 days.¹² The foregoing results are consistent with the suppression or eradication of demodectic mites, establishing piroctone olamine treatment as anti-demodectic, as shown by the reduction or elimination of blepharitis, meibomian gland dysfunction, and papules about the eyelids, nose, lips, and lip margins.

The meibomian glands, and the face around the eyes and nose, contain the most oily skin and hair on the body, consistent with demodex's primary metabolic necessity, body sebum, and also consistent with type 2 acne rosacea (papulopustular or granulomatous rosacea), found predominantly on the oily skin of the nose, forehead, chin, and cheeks. Demodex is considered the “missing link” to comprehending the causative agent of type 2 acne rosacea.^{13 14 15}

Type 1 flushing rosacea involves flushing bright red skin and broken blood vessels on the face, but many or most people with type 1 rosacea progress from flushing and broken blood vessels on the face to type 2 acne rosacea. Type 2 acne rosacea has all the signs of type 1 rosacea, plus papules and pustules that have been called “the gravestone of dead Demodex.”¹⁶ Type 4 ocular rosacea has symptoms and signs identical to blepharitis and meibomian gland dysfunction, but is diagnosed when additional cutaneous signs and symptoms of rosacea are also present—for example, facial flushing, or periocular erythema, or rosacea papules and pustules presenting as a slow growing bump from the occlusion of a pilosebaceous unit, or an occluded meibomian gland (chalazion), or a pimple-like staphylococcal infection manifested as a stye (hordeolum). Type 4 ocular roasacea, blepharitis and meibomian gland dysfunction have numerous identical signs and symptoms, such as dry eyes, red eyes, red eyelid margins, crusting of the eyelids, light sensitivity, itching, foreign body sensation, lash fallout, misdirected lashes, pinholes in the cornea, corneal ulceration in severe longstanding cases, or any combination of these signs and symptoms.

The International Workshop on Meibomian Gland Dysfunction: Report of the Definition and Classification Subcommittee stated that “obstructive MGD [meibomian gland dysfunction] is the most pervasive,” of eye disorders, and, “obstructive MGD may be seen in Sjögren's syndrome, seborrheic dermatitis, acne rosacea, atopy, and psoriasis.” The Subcommitte's most recent medical classification system for eye irritation is quoted below:
Blepharitis is a general term describing inflammation of the lid as a whole; marginal blepharitis is inflammation of the lid margin and includes both anterior and posterior blepharitis.
Anterior blepharitis describes an inflammation of the lid margin anterior to the gray line and concentrated around the lashes. It may be accompanied by squamous debris or collarettes around the lashes, and inflammation may spill onto the posterior lid margin.
Posterior blepharitis describes an inflammation of the posterior lid margin, which may have different causes, including MGD, conjunctival inflammation (allergic or infective), and/or other conditions, such as acne rosacea.
MGD describes a chronic, diffuse abnormality of the meibomian glands, commonly characterized by terminal duct obstruction and/or qualitative/quantitative changes in the glandular secretion. It may result in alteration of the tear film, symptoms of eye irritation, clinically apparent inflammation, and ocular surface disease.¹⁷

Demodex do not have an excretory opening. When demodex die, they decompose and release Bacillus oleronius, the primary sensitizing component of a demodex reaction in people with rosacea. Type 2 acne rosacea is currently treated with topical and systemic antibiotics, even though antibiotics do not kill demodex and treat only the secondary infection and inflammation caused by the release of B. oleronius and other detritus associated with dead and decaying demodex.

Nasal congestion is a common complaint among people with rosacea. Decomposing demodectic mites in and around the skin, eyes, eyelashes and meibomian glands produce a variety of fine organic particles that provide metabolic components for other microbes in the skin, eyes, nasal passages and sinuses, in both humans and non-human mammals. 18 19 20 21 22 Demodex mouth-part chewing and mechanical movement through the sebaceous glands, hair follicles, and meibomian glands can cause severe skin and eye irritation. Demodectic mites' persistent cutting and feeding on the epithelium of hair follicles and main collecting tubules of the meibomian glands, and their secretions, excretions, and somatic debris, result in inflammation and dilation of the sinuses (sinusitis) and nasal passages (rhinitis), which pave the way for active or passive introduction of pathogenic bacteria. A microbial food chain or cycle thereby takes place throughout the sebaceous glands, hair follicles, skin, and meibomian glands involving detritus feeders (detritivores) and the micro-organisms that multiply on them. The nasolacrimal ducts drain from the eyes into the nasal sinus cavities, allowing demodex and inflammatory detritus from demodex to invade the sinuses, resulting in whole or in part, in an inflammatory response to demodex, along with sinus congestion. Neurogenic inflammation and the individual hypersensitivity characteristics of blepharitis, meibomian gland dysfunction, type 1 flushing rosacea, type 2 acne rosacea, type 4 ocular rosacea, demodicosis gravis, granulomatous rosacea, and pityriasis folliculorum, are induced, in whole or in part, by demodex and demodex detritus originating on the skin and eyes, and in the nose and sinuses, which is prevented and treated by diminishing or eradicating the demodex and the demodectic source of the detritus cycle, by use of the compositions comprising the invention.

After compounding and testing the compositions comprising the invention, piroctone olamine was identified as an effective anti-demodectic treatment. With once-daily, at night, topical application, of a rinse-off shampoo/shower gel, and of a leave-on skin lotion, rapid and dramatic reduction occurred in the symptoms and signs of blepharitis, meibomian gland dysfunction, and reduction of papules around the margins of the eyes and the lips. The compositions comprising the invention containing piroctone olamine, and in some embodiments, climbazole, have a significant advantage over the use of antibiotics. The compositions comprising the invention suppress or eradicate demodex, the primary causative agent of blepharitis, demodicosis, type 1 rosacea, type 2 acne rosacea, type 4 ocular roasacea, meibomian gland dysfunction, pityriasis folliculorum, granulomatous rosacea, demodicosis gravis, chronic conjunctivitis, allergic conjunctivitis, trichiasis, perioral dermatitis, and in non-human mammals, demodectic mange. The compositions comprising the invention are novel, and have not previously been reported or used to prevent or treat the foregoing skin and eye disorders.²³

Veterinary doctors are certain that demodex are the causative agent of demodectic mange (demodicosis) in mammals. See, non-patent references 18, 19, 20, 21, and 22. The anti-demodectic compositions described herein are effective treatments for the symptoms of demodectic mange, including alopecia and pruritus, in non-human mammals. The compositions comprising the invention are novel, and have not previously been reported or used to prevent or treat demodectic mange. Demodectic species that can be controlled by the compositions described herein include, but are not limited to, D. folicularum, D. brevis, D. canis, D. gatoi, D. bovis, D. equi, D. ovis, D. cati, D. phyloides, and D. caprae. The patient or subject to be treated with the compositions and methods described herein can be any mammal, including but not limited to, a dog, a cat, a horse, a cow, a sheep, or a pig, suffering from demodectic mange.

Clariant, a manufacturer of piroctone olamine, does not describe piroctone olamine as having any effect on demodex, blepharitis, meibomian gland dysfunction, or any of the subtypes of rosacea. In Clariant's filing with the FDA, the claim is that piroctone olamine is “to relieve or control dandruff, seborrheic dermatitis, and/or psoriasis.”²⁴ Clariant's sales brochure for piroctone olamine shows the antifungal and antibacterial activity of piroctone olamine, but does not describe piroctone olamine as an aracacide or mitacide, or as having any effect on demodex, blepharitis, meibomian gland dysfunction or any of the subtypes of rosacea.²⁵ Clariant's above-cited FDA filing and sales brochure show that piroctone olamine is non-toxic in topical usage thousands of time in excess of the compositions disclosed in this patent application. Clariant's above-cited FDA filing and sales brochure illustrate that the compositions described herein have novel functions.

Demodex are ubiquitous in the human biome, and so re-infestation is inevitable and continuing treatment is necessary. Accordingly, the compositions comprising the invention are not a total cure for a chronic condition, most likely an innate immune response or allergic reaction to demodex, but instead constitute effective treatments for the symptoms that some humans, and some mammals, experience to infestations or an overload of demodectic mites.

### Combination Treatments

Both piroctone olamine and climbazole have the current status of Pending Monographs of the United States Pharmacopeial Convention (USP), and neither are allowed for over the counter compositions sold in the US or Canada. There are no reports, articles, or clinical trials of piroctone olamine as an anti-demodectic treatment. Piroctone olamine has been approved for cosmetic use in the European Union since approximately 1985 and is found in over the counter topical compositions used to treat dandruff, psoriasis and eczema.²⁶

Climbazole provides an anti-fungal component to the composition, to dissolve the cell walls of Malassezia and decrease the fungal load on skin, working synergistically with piroctone olamine. Climbazole treats the symptoms of seborrheic dermatitis, which are believed to result from an underlying weakness of the skin barrier that allows Malassezia yeast and hyphae forms (dimorphic fungal growth) to flourish, causing itchy, flat, red lesions to spread across the body. Up to 0.5% climbazole face/scalp leave-on lotion and 2% climbazole rinse-off full body shampoo are authorized in the European Union as safe, over the counter, cosmetics for humans.²⁷ With respect to European Union (“EU”) authorization for a climbazole-based face/scalp lotion however, the head, plus the hands for application of the lotion onto the face and scalp, comprise a fraction of the total surface area of skin on the average human body. The head is approximately 8.5% of the total human body surface area, and each hand is approximately 2.4% of the total, and so, the body surface area authorization for 0.5% climbazole, as stated by the EU committee, is 2,400/18,000, which is 13.3% of the entire body. Therefore, by interpolation of EU standards, a full body leave-on lotion, in the embodiment including climbazole, is preferentially compounded at 0.07% (and effective even at concentrations of 0.03 to 0.11%) climbazole. Likewise, the embodiment of the full-body rinse-off shampoo/shower gel that adds climbazole is also preferentially compounded at 0.07%, in order to comply with the EU's safety margin for climbazole-based cosmetics even if the rinse-off product is misused by the patient as a leave-on product. Nevertheless, when the leave-on lotion is limited to use as a scalp and face lotion, this embodiment of the composition preferentially can have a concentration of up to 0.49% climbazole, and when the rinse-off shampoo composition is limited to use as a scalp and face shampoo, this embodiment of the composition preferentially can have a concentration of up to 0.49% climbazole. Climbazole is the most effective, currently available compound that reduces an overload of Malassezia.²⁸ Solutions as low as 0.01% of either/both climbazole and/or piroctone olamine may be effective.

Malassezia and demodex are commensal, and both are specialized to metabolize body sebum, and reside in or near the pilosebaceous units. Because these and other micro biotic constituents of the skin live in competition with each other, the embodiment of the composition with climbazole prevents Malassezia from taking advantage of the decrease in demodex populations and the increase in available food and living spaces that result from topical compositions containing piroctone olamine.²⁹ The preferred ratio of piroctone lamine to climbazole is 2:1. This ratio may be exact, but it is generally known to be useful as approximately 2:1, meaning it is effective at ratios of 1:2, 1:1, and as much as 4:1.

The rinse-off cleanser and a leave-on lotion of the compositions comprising the invention containing piroctone olamine, and climbazole in some embodiments, function synergistically with caprylic/capric oils, commonly referred to as medium chain triglycerides or “MCT oil”, because Malassezia fungi are not able to metabolize oils that have carbon chain lengths less than 11.³⁰ Malassezia and demodex co-exist on the human body and seek out the same sebum sources, such that treating one creates conditions where the other can become numerous and pathogenic, and therefore both must be synergistically suppressed, to treat blepharitis, meibomian gland dysfunction, and the subtypes of rosacea. The effective medicinal uses are best used with a base-oil with a carbon chain of less than 11, such as a C10. MCT oil (caprylic/capric oil), can be comprised only of carbon chain lengths of C8 and C10. Fractionated coconut oil is another useful base-oil, similar to MCT oil. Other oils with small (i.e. less than C8) or (C8 to C10, or as known in the art, medium chain triglycerides) are useful as base oils.

Piroctone olamine and climbazole are barely soluble in water, in concentrations much too low to be useful.³¹ Each can be dissolved in sodium lauryl sulfate, but that is a harsh anionic surfactant and a known skin irritant, currently unacceptable to substantial numbers of consumers, and cannot be used around the margins of the eyes. Moreover, sodium lauryl sulfate surfactants cannot be compounded into a leave-on lotion because it must always be washed off to prevent skin irritation. However, piroctone olamine powder and climbazole powder dissolve in 4-5 parts ethanol and/or isopropanol, weight to volume, when heated to 170 degrees F., and that solution easily dissolves in oil at approximately 170 degrees F., and when the proper emulsifiers are added to the oil-admixture, all the components thereof easily emulsify in water at approximately 170 degrees F. With this innovation, piroctone olamine and climbazole can be added to any topical aqueous composition. An additional innovative advantage of this multi-step method for dissolving piroctone olamine and/or climbazole, and mixing into oil, and then emulsified with water, is that a lotion can be compounded, in addition to a surfactant-based cleanser. Moreover, skin, hair, and hair follicles are somewhat oil soluble and barely water soluble. Employing an oil in water emulsion improved the delivery of an effective dose of active ingredients onto the skin, hair, and hair follicles, and allows relatively low concentrations of the active ingredients to be effective. In addition, the oil-emulsion composition described herein is non-irritating to the corneas, and to the thin, sensitive skin around the margins of the eyes.

The rinse-off cleanser of the compositions comprising the invention containing piroctone olamine, plus climbazole in other embodiments, is preferentially compounded with nonionic and/or amphoteric or zwitterionic surfactants in the emulsion to remove environmental dirt, microorganisms, and sebum from the skin surface, while the oil-component of the emulsion includes the active ingredients, which remain suspended in the lather during the cleansing process. During lathering, the surfactant lather becomes diluted with water and the emulsion of oil-plus-active ingredients breaks, providing a synergistic advantage, depositing the active ingredients onto environmentally clean, moist skin, resulting in improved active ingredient penetration to the stratum corneum, hair follicles, sebaceous glands, and barrier-layer lipid structures of the skin. By comparison, anionic surfactants are irritating to the skin and eyes. Eye irritation does not occur when the surfactants are: nonionic, preferentially, but not by way of limitation, decyl glucoside and poly/glucose lactylate; and, amphoteric or zwitterionic surfactants, preferentially, but not by way of limitation, coco-betaine (cocamidopropyl betaine). Nonionic and amphoteric or zwitterionic surfactants are sufficiently mild and additional eye irritation will not result when lathering the lid-margins of the eyes.

The rinse-off cleanser of the compositions comprising the invention containing piroctone olamine, and with climbazole in some embodiments, functions synergistically with the properties of the stratum corneum, the dead and dry outer layer of epidermis. The outermost layers of the stratum corneum double in thickness when wet. When the outermost layers swell via hydration and then shrink via inevitable dehydration, additional mechanical stress is place on microbial biofilms that occupy the surface of the skin. The oil in water emulsion of the present invention is an advantageous innovation over other products, such as topical ivermectin or tea tree oil, that are not used in the process of full-body lathering while taking a shower. Treating the entire body, when wet, reduces or eradicates the detritus cycle from the sinuses and nasal passages, and increases the efficacy of the active ingredients described herein.

The rinse-off cleanser of the compositions comprising the invention, is used as a typical topical shampoo/shower gel, and can be used over the entire scalp and body, with the exception that the rinse-off cleanser is applied to a wet body and lathered without running shower-water, so that the cleanser can be massaged onto the skin and scalp for 3 to 5 minutes, paying particular attention to the face and margins of the eyelids, and then rinsed off. Neither composition is intended to be applied directly onto the cornea, as the eyes are expected to be shut when applying either composition.

The leave-on lotion of the compositions comprising the invention allows the active ingredients to remain on the skin during sleep, when demodex are most active and demodex males are exposed on the skin surface. The leave-on lotion described herein is used as any typical lotion, and can be used over the entire body, paying particular attention to the face and margins of the eyes, preferentially at night. By application to the entire body, demodex can be reduced or eradicated, and the male demodex cannot temporarily escape from a small application site, e.g., spot treatments of the face or the margins of the eyes, the method of other current treatments.

### INCORPORATION BY REFERENCE

All publications, patents, and patent applications cited herein are incorporated by reference in their entirety to the same extent as if each individual publication, patent, or patent application was specifically and individually indicated to be incorporated by reference. Discussion of documents, acts, materials, devices, articles, or the like which have been included in the present specification is for the purpose of providing context for the present invention. Such discussion is not an admission that any or all of these matters form part of the prior art with respect to any inventions disclosed or claimed.

### PRIOR ART

Current compositions for the topical prevention and treatment of the inflammatory response to demodex are difficult to use or have less effectiveness than the compositions comprising the invention. The current best-practices medical office treatment is topical application, via eyelid scrubs, of 5% to 50% tea tree oil.³² However, tea tree oil is a burning, volatile, essential oil, and even when diluted to 20%, a doctor must carefully apply it to the margins of the eyelids, scrub the lids several times over the course of thirty minutes, and then wash it off. Self-administered home treatments with even as little as 5% tea tree oil are difficult because the eyes must remain tightly closed while applying tea tree oil. The slightest accidental opening of the eyes allows tea tree oil to seep past the eyelids and burn the cornea. As of Dec. 23, 2014, a once-daily prescription topical 1% ivermectin face-cream, Soolantra, has been approved by the FDA for treating acne rosacea, which is relevant to this patent application because Soolantra is described as having anti-demodectic properties. Soolantra's parent company's patent applications disclose that ivermectin-based compositions treat rosacea, blepharitis and ocular rosacea caused by demodectic mites (US 2011/0274631 A, and US 2012/0053140 A1). However, Soolantra's prescribing information states that it must not be used near the eyes, making it useless to treat ocular rosacea. Ivermectin is also the active ingredient in Sklice, a prescription treatment that discloses effectiveness against head lice, which is relevant to this patent application because head lice are arachnids, the same family as demodex (US 2000/6103248). Metronidazole, topical or systemic, is an antibiotic often prescribed for rosacea, and its mechanism is disclosed as treating the bacterial by-products of demodex. Doxycycline (Oracea) is tetracycline antibiotic often prescribed for rosacea that has a similar mechanism. Other, less-used, less-effective treatments include yellow mercurial ointment, sulfur ointment, camphorated oil, crotamiton, choline esterase inhibitors, sulfacetamide (Plexion, Rosac, Rosanil, Rosula, or Sulfacet-R), and corticosteroids.

Clairant's patent application for piroctone olamine (Octopirox) discloses effectiveness in cosmetic applications against bacteria, yeasts, and fungi (US 2002/0037299 A1). No mention of effectiveness against mites, arachnids, or arthropods is made. Compositions containing piroctone olamine at concentrations of 0.5% to 1.5% have been disclosed as treating pruritus (itchy skin) and seborrheic dermatitis, in a prescription-only lotion sold as Promiseb (US 2011/0229417 A1). Numerous other patents disclose cosmetic compositions including piroctone olamine, but all are limited to treating pruritus, dandruff and/or seborrheic dermatitis, and none describe piroctone olamine as anti-demodectic or as miticidal, or for treating blepharitis, meibomian gland dysfunction, the subtypes of rosacea, pityriasis folliculorum, demodicosis gravis, granulomatous rosacea, perioral dermatitis, chronic conjunctivitis, allergic conjunctivitis, or demodectic mange (demodicosis) in mammals. For example, a composition for treating dandruff containing climbazole, zinc pyrithione and piroctone olamine, is found at US 2002/0172648 A1. A composition for treating dandruff is disclosed as a low pH shampoo containing climbazole, found at US 1989/4867971. Compositions containing tea tree oil and/or certain chemical constituents of tea tree oil have been disclosed to treat blepharitis (US 2010/0273870 A1, US 2010/0273870 A1). Compositions containing ivermectin have been disclosed to treat rosacea (US 1999/5952372 A).

The prior art that has piroctone olamine in hair and skin cleansers employs as a solvent, sodium lauryl sulfate, a very common cosmetic-surfactant that can be irritating to the skin and cornea, even in normal use as a rinse-off cleanser.^{33 34} Sodium lauryl sulfate is well-known to be irritating to skin if not completely rinsed off. Prior art does not disclose a non-irritating solution to the water insolubility problem presented by piroctone olamine and climbazole, as disclosed herein. Because the openings of the meibomian glands contact the cornea, compositions that treat demodectic blepharitis, ocular rosacea, and meibomian gland dysfunction must be non-irritating to the cornea to be functional. The patent claims and methods submitted in this application are novel, functional, and advantageous.

### Mode of Operation: Methods and Examples of Compositions for Topical Application.

The topical compositions herein are used each time the patient bathes or wets all or part of their skin and hair, and are preferably used once daily. The topical compositions herein are used as a body cleanser and shampoo, and after drying, as a topical skin lotion, preferably sequentially and consecutively. Multi-treatments per week, i.e., at least every 3 days, are preferable.

With quadruped mammal patients (which may also include bipedal non-humans), effective treatments for demodectic mange, described herein, are preferred to be applied daily, and if not, every time after the animal's skin becomes wetted, until signs of demodicosis are not visible.

Effective treatments with shampoo and shower gel should be applied by wetting the hair and skin, applying an effective amount to the hair and skin (as well as scalp and/or skin areas under hair), lathering, and rinsing. Effective treatments are preferentially used over a large area of the body, such as more than 10% or 25%, as much as 50%, and preferably as close to 75% to 100% of the exposed skin and hair as possible. Dry-skin application of the lotion is the preferred treatment. If post-bathing skin-wetting occurs, or when the patient or animal otherwise becomes wet, the lotion is preferentially applied after drying. Treatment should be included for at least 23 days, or 24 days, or one month, or preferentially four months, or may be extended as long as signs or symptoms persist.

### Summary of Test Results, Unpublished.

On day-23 of once per day use of the rinse-off shampoo/shower gel comprising the invention containing 0.14% topical piroctone olamine and 0.07% climbazole, the inventor was surprised to notice his red, dry, itchy eyes, and crusting eyelash symptoms suddenly began to resolve favorably and papules around the eyes and lips began to shrink. Demodex are believed to have a maximum lifespan of 23 days. Medical research confirmed ocular demodicosis is an inflammatory response to Demodex. On day 24 the leave-on lotion comprising the invention was compounded and used once per day. From days 25-40, tear production continually increased, along with significant shedding of cylindrical dandruff and waxy, crusty debris from the eyelashes. From days 40-45, the meibomian glands opened up and thick oily deposits were gently expressed with pressure on the outer margins of the eyelids. Two significant points in the timeline for clearing of symptoms most likely occur because D. folliculorum lives near the surface of the stratum corneum at the openings of the hair follicles, whereas D. brevis lives deep in the sebaceous glands, such that the expression of detritus created by D. brevis occurred more slowly because the sebaceous glands are significantly deeper than the openings of the hair follicles. From days 45-100, hard, tiny, dot-like particles at the surface of the eyelashes scraped off, tear production greatly increased, dry eye symptoms receded, and red eyelid symptoms vanished. From days 60-100, swollen eyelids slowly shrank to normal, red eye symptoms receded, and papules around the lips, corners of the eyes, nose, and lips become pustules that broke, bled slightly, scabbed over, and healed. By day 110, signs and symptoms of blepharitis and meibomian gland dysfunction were completely gone, and only two formerly prominent papules remained, but barely visible and only on close inspection. Dramatic healing of ocular irritation and skin inflammation occurred during treatment with the compositions comprising the invention, which are novel in their scope and utility.

A brief history underscores the relevance of the test results. The inventor, now 61 years of age, had exhibited signs of severe seborrheic dermatitis since age 5, eventually covering his entire body, consisting of oily, adherent but flaky-appearing skin, and extremely itchy, red, flat, lesion-like bumps (annular plaques) about 1 mm in height, and 5 to 20 mm in diameter. Dermatitis caused a severe impairment in the quality of life. Therefore, beginning three years ago, the inventor studied dermal inflammations, to conduct an accurate differential diagnosis and treat his skin condition. The only commercial product that provided significant relief from dermal disorders was a 2% climbazole shampoo, made by Hegor and sold in Europe. Hegor's product however contains sodium lauryl sulfate, which irritates the skin and so this shampoo cannot be used as a leave-on lotion. Moreover, all commercial lotions are formulated with oils with carbon-chain lengths longer than 10 such that Malassezia metabolizes all commercial lotions, which greatly exacerbated the inventor's seborrheic dermatitis. (See, non-patent reference number 30, by Wilde.) Dissatisfied with commercial products that made seborrheic skin conditions worse, sixteen months ago the inventor compounded topical compositions, a rinse-off shampoo/shower gel and a leave-on lotion, where the active ingredient was 0.07% climbazole and the base-oil was exclusively MCT oil (caprylic/capric oil), comprised only of carbon chain lengths of C8 and C10. While using this compounded topical treatment, all symptoms of seborrheic dermatitis induced by Malassezia were eliminated approximately one year ago. However, after seborrheic dermatitis symptoms were eliminated, red eye and dry eye symptoms increased significantly, and the inventor was unable to wear contact lenses for 8 months previous to compounding the topical compositions described herein, which is consistent with Demodex and Malassezia competing for sebum sources on the body. Prior clearing of seborrheic dermatitis induced by Malassezia is relevant to the invention described herein, because of the interim phase of suffering from ever increasing signs and symptoms of blepharitis, meibomian gland dysfunction and papules about the lips, lip margins, eyelid margins, and nose. Daily use of the topical compositions is preferred, although use of the compositions at a longer interval, each time the skin or scalp is wetted, appears to produce similar results. Use of the compositions comprising the invention with 0.14% topical piroctome olamine, plus 0.07% climbazole, has increased utility, and piroctone olamine is the anti-demodectic treatment.

Presented below are two examples of the formulary ingredients used in the topical compositions containing piroctone olamine, and in some embodiments, climbazole. The following specific examples are intended as illustrative and not limitative. All ingredients are shown as a percentage by weight.

Piroctone Olamine + Climbazole Lotion

| Product | % |
|---|---|
| Distilled Water (water) | 70.3% |
| Caprylic/Capric Oil (oil) | 15.2% |
| Vegetable Glycerin (humectant) | 2.25% |
| Niacinamide (vitamin) | 1.9% |
| Xylitol (optional) | 1.89% |
| Cetearyl Alcohol 30/70 NF (emulsifier) | 1.8% |
| Glyceryl Stearate (emulsifier) | 1.41% |
| dl-Panthenol (vitamin) | 1.3% |
| Propylene Glycol (stabilizer) | 1.0% |
| Ethoxydiglycol (stabilizer) | 0.6% |
| Optiphen ND (preservative) | 0.5% |
| Ethanol, undenatured, absolute (solvent) | 0.4% |
| Ceteareth 20 (emulsifier) | 0.3895% |
| Hydrolyzed Collagen (optional) | 0.36% |
| Isopropanol (solvent) | 0.3% |
| Piroctone Olamine | 0.14% |
| Propylene Glycol Alginate (hydro-colloid) | 0.09% |
| Climbazole | 0.07% |
| Citric Acid (pH adjuster) | 0.05% |
| PEG-7 Glyceryl Cocoate (emulsifier) | 0.05% |
| | 100.00% |

Piroctone Olamine + Climbazole Shampoo/Shower Gel

| Product | % |
|---|---|
| Distilled Water (water) | 65.85% |
| Coco-Betaine (surfactant) | 12.5% |
| Decyl Glucoside (surfactant) | 6.25% |
| PolyGlucose/Lactylate (surfactant) | 6.0% |
| Vegetable Glycerin (humectant) | 2.5% |
| Caprylic/Capric Oil (oil) | 1.6% |
| Xylitol (humectant) | 0.8% |
| Niacinamide (pro vitamin) | 0.6% |
| Hydroxypropyl Methylcellulose, (hydro-colloid) | 0.5% |
| Optiphen ND (preservative) | 0.5% |
| dl-Panthenol (pro vitamin) | 0.5% |
| Ethanol, undenatured, absolute (solvent) | 0.4% |
| Isopropanol (solvent) | 0.3% |
| Hydroxypropyl Starch (optional) | 0.3% |
| Di-PPG-2-Myreth-10-Adipate (emulsifier) | 0.21% |
| Ethoxydiglycol (stabilizer) | 0.2% |
| Sclerotium Gum (Hydro-gel) | 0.2% |
| Xanthan Gum (Hydro-gel) | 0.18% |
| Piroctone Olamine | 0.14% |
| Sodium Carbomer (Hydro-gel) | 0.13% |
| PEG-7 Glyceryl Cocoate (emulsifier) | 0.1% |
| Climbazole | 0.07% |
| Citric Acid (pH adjuster) | 0.06% |
| Dimethicone 350 (oil) | 0.06% |
| Silk Amino Acids, Blue FD&C (optional) | 0.05% |
| | 100.00% |

Methods for compounding the topical skin lotion or the skin and hair shampoo/shower gel compositions comprising the invention include the oils+emulsifiers to be separately heated to approximately 170 degrees F., and the piroctone olamine, and climbazole, to be combined with the solvents and separately heated to approximately 170 degrees F., and then stirred briefly into the oils+emulsifiers, before stirring the oils+emulsifiers+piroctone olamine, and climbazole, into a water phase. This method of the invention completely solubilizes and emulsifies climbazole and piroctone olamine, such that anionic surfactant-solvents are unnecessary and relatively small concentrations of the active ingredients are therapeutically effective.

To compound the topical skin lotion comprising the invention, the heated oils+emulsifiers+piroctone olamine and climbazole, are combined and stirred, and then poured into the heated water, and then the hydro-colloid (which has been previously suspended in the humectant and stabilizers, for ease of pouring and avoidance of clumping) is blended into the small vortex of a blender containing the oil and water emulsion. During the cool-down phase, at or below approximately 120 degrees F., addition of the pH adjuster, pro vitamins, optional ingredients and preservative occurs.

To compound the topical skin and hair shampoo/shower gel comprising the invention, to one-half of the total amount of the water, the hydro-gels are added before heating, and then the water is heated to 170 degrees F., and during the cool-down phase, at or below approximately 120 degrees F., stir in the pH adjuster, pro vitamins, optional ingredients and preservative, and then at approximately 70 degrees F. add the polyglucose/lactylate, decyl glucoside and coco-betaine surfactants, hereinafter collectively called “1^{st} component.” To one-quarter of the total amount of water, after heating to approximately 170 degrees F., add into the small vortex of the heated water in a blender, the hydro-colloid, which was previously suspended in the humectant and the stabilizer for ease of pouring and avoidance of clumping, hereinafter collectively called “hydro-colloid component.” After heating the remaining one-quarter of the total amount of water to approximately 170 degrees F., add into the small vortex of the heated water in a blender, the oils+emulsifiers+piroctone olamine, and climbazole, and to that after cooling to approximately 70 degrees F., add the water+hydro-colloid component, and then add that to the 1^{st} component.

Aside from novel use of the active ingredients piroctone olamine and climbazole, and solubilized active ingredients in an oil in water emulsion, the remaining ingredients typical to topical aqueous lotions and skin or hair cleansers are obvious, and therefore the composition of the invention also comprises any suitable oil or oils derived from mineral, plant, fruit, seed, nut, or vegetable, although preferably caprylic/capric oil, and any additional suitable humectants, hydro-colloids, hydro-gels, surfactants, emulsifiers, thickeners, pro-vitamins and optional ingredients, and similar products, or variations of all the products listed in the tables above, in suitable proportions, which are used or known to persons skilled in the art, in order to obtain a topical composition that may be used according to the invention, in the form of a cream, lotion, shampoo, shower gel, spray, lotion, pomade, or oil, in the form of an oil in water emulsion, and additional products or methods known to persons skilled in the art to preserve, improve, modify or stabilize the composition from a cosmetic point of view.

As described throughout this specification, the ingredients in compositions of the present invention can be present within the compositions in a variety of amounts. The amounts can be measured by total weight or volume of the composition, but preferentially by weight. By way of example only, an ingredient can be included into the composition at 0.0001, 0.001, 0.01, 0.1, 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99%, or more, or any range or integer derivable therein, by weight or volume of the total composition. The ratio of any ingredient within the composition when compared to another ingredient can be from about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 50:1, 60:1, 70:1, 80:1, 90:1, 100:1, or more or any number derivable therein, by weight or volume of the total composition.

Topical compositions formulated to treat demodectic mange in non-human mammals may include climbazole, but after treatment the animal may be fitted with a cone shaped plastic “Elizabethan collar” or an inflatable collar around the neck, because all animals, and particularly animals with demodectic mange, gnaw at sores and lick their bodies repeatedly as an instinctive grooming method. EU cosmetic safety factors for climbazole are modeled on human behavior, and these safety factors do not allow for climbazole-ingestion via gnawing and licking. Therefore, climbazole may be deleted from the compositions in circumstances where it is impractical for the animal to be collared or muzzled.

### REFERENCES

All references listed below are also e-filed with the Information Disclosure Statement.

### US Patent and Patent Application Publications

1. US 2011/0274631 A1, Nov. 10, 2011, Kaoukhov, Avermectin/metronidazole compositions for treating afflictions of the skin, e.g., rosacea.
2. US 2012/0053140 A1, Mar. 1, 2012, Kaoukhov, Administration of avermectin/milbemycin compounds for the treatment of ophthalmic pathologies.
3. US 2000/6103248 A, Aug. 15, 2000, Burkhart, Carrier containing a surfactant effective to allow the topical preparation to be washed out of the scalp hair, and the carrier having a viscosity within a range of from about 25,000 centipoise to about 85,000 centipoise at 21 degrees c.
4. US 2002/0037299 A1, Mar. 28, 2002, Turowski-Wanke, Cosmetic compositions comprising zinc salts of piroctone olamine.
5. US 2011/0229417 A1, Gurge, Sep. 22, 2011, Emollient Foams for Treatment of Seborrheic Dermatitis.
6. US 2002/0172648 A1, Hehner, Nov. 21, 2002, Can be applied to a hair-covered body surface to eliminate or alleviate existing dandruff or to prevent or reduce occurrences of dandruff.
7. U.S. Pat. No. 4,867,971 A, Colgate-Palmolive Company, Sep. 19, 1989, Antidandruff.
8. US2014/8865233, Aug. 29, 2013, Gao, Compositions and methods for treating Demodex infestations.
9. US 2010/0273870 A1, Oct. 28, 2010, Gao, Compositions and methods for treating demodex infestations
10. US 1999/U.S. Pat. No. 5,952,372 A, Sep. 14, 1999, McDaniel, Oral administration or topically applying ivermectin in a dosage to eliminate demodex folliculorum mites from hair follicles in affected skin.

### Non-Patent Publications Cited

¹ ROBLEDO, et al., Hypothesis of demodicidosis rosacea flushing etiopathogenesis. Med. Hypotheses (2015), accepted Jan. 22, 2015, draft available online before printing, © 2015 Elsevier Ltd, Columbia/USA.doi.org/10.1016/j.mehy.2015.01.036.
² LACEY, et al., Under the lash, Demodex mites in human diseases, Biochem (Lond), Aug. 1, 2009; 31(4): 2-6, United Kingdom.
³ KIM, et al., Clinical and immunological responses in ocular demodecosis, J. Korean Med. Sci. 2011, 26:1231-1237, Korea. doi.org/10.3346/jkms.2011.26.9.1231.
⁴ LI, et al., Correlation between ocular Demodex infestation and serum immunoreactivity to bacillus proteins in patients with facial rosacea, © 2010 by the American Academy of Ophthalmology ISSN 0161-6420/10, 1-8, Published by Elsevier Inc., USA. doi:10.1016/j.ophtha.2009.09.057.
⁵ KHEIRKHAH, et al., Fluorescein dye improves microscopic evaluation and counting of demodex in blepharitis, with cylindrical dandruff, Cornea, Volume 26, Number 6, July 2007, © Lippincott Williams & Wilkins, USA.
⁶ FORTON, et al., Demodicosis and rosacea: epidemiology and significance in daily dermatological practice, J. Am. Acad. Dermatol., 2005; 52: 74-87, USA. © Elsevier.
⁷ ABELSON, et al., Eavesdropping on blepharitis—once bacteria start talking, the news they share may turn out to be bad, Review of Ophthalmology, Oct. 18, 2007, USA.
⁸ BHANDARI, et al., Blepharitis: Always Remember Demodex, Middle East Afr. J. Ophthalmol., 2014 October-December; 21(4): 317-320, December, 2014, India. doi: 10.4103/0974-9233.142268.
⁹ LIU, et al., Pathogenic role of Demodex mites in blepharitis, Curr. Opin. Allergy Clin. Immunol., 2010 October; 10(5): 505-510, Oct. 1, 2010, © 2010 Wolters Kluwer Health 1 Lippincott Williams & Wilkins, USA. doi:10.1097/ACI.0b013e32833df9f4.
¹⁰ HSU, et al., Demodicosis: A clinicopathological study, J. Am. Acad. Dermatol., March 2009, 453-462, Dermatopathology, © 2008 by the American Academy of Dermatology, Inc. (USA). doi:10.1016/j.jaad.2008.10.058.
¹¹ STEPHENSON, Blepharitis diagnosis: don't forget Demodex, Review of Ophthalmology, Sep. 6, 2012, USA.
¹² KNOP et al., The International Workshop on meibomian gland dysfunction: Report of the Subcommittee on anatomy, physiology, and pathophysiology of the meibomian gland, investigative ophthalmology & visual science, Special Issue 2011, Vol. 52, No. 4, pp. 1938-1978, at p. 1966, USA. doi:10.1167/iovs.10-6997c.
¹³ FORTON, Papulopustular rosacea, skin immunity and Demodex: pityriasis folliculorum as a missing link, JEADV 2012, 26, 19-28, Journal of the European Academy of Dermatology and Venereology, Germany. DOI: 10.1111/j.1468-3083.2011.04310.x JEADV.
¹⁴ CZEPITA, et al., Demodex folliculorum and Demodex brevis as cause of chronic marginal blepharitis, Annales Academiae Medicae Stetinensis, 2007, 53, 1, 63-67, Poland.
¹⁵ JARMUDA, et al., Potential role of Demodex mites and bacteria in the induction of rosacea, Journal of Medical Microbiology (2012), 61, 1504-1510, United Kingdom. DOI 10.1099/jmm.0.048090-0.
¹⁶ JESITUS, Research suggests rosacea subtypes may be different conditions, Dermatology Times, Dec. 1, 2012, pp. 28-30, at p. 30, quoting Frank C. Powell, M.D., addressing 2012 annual meeting of the American Academy of Dermatology.
¹⁷ NELSON, et al., Quoted from Table 2, The International Workshop on Meibomian Gland Dysfunction: Report of the Definition and Classification Subcommittee, Investigative Ophthalmology & Visual Science, Special Issue 2011, Vol. 52, No. 4, pp. 1930-1937, USA. doi:10.1167/iovs.10-6997b.
¹⁸ ABU-SAMRA, et al., A study on the nature of association between Demodex mites and bacteria involved in skin and meibomian gland lesions of demodectic mange in cattle, Vet. Med. Int., Aug. 6, 2014: 413719, Nigeria/USA. doi: 10.1155/2014/413719.
¹⁹ ABU-SAMRA, Meibomian gland demodicosis in cattle: The clinical disease and diagnosis. Inter. J. Vet. Sci, 3(1): 11-17, Nigeria/USA.
²⁰ ABU-SAMRA, Bovine demodicosis: prevalence, clinico-pathological and diagnostic study, J. Vet. Adv., 2014, 4(2): 381-397, Nigeria/USA.
²¹ NASH, Diagnosing Demodex infection, Banfield Journal, March/April 2006, 22-27, USA.
²² RUTAN, Treating canine Demodicosis, Banfield Journal, March/April 2006, 30-38, USA.
²³ MEDEDICUS LLC, Updates in ocular surface wellness: part 2, dry eyes, Review of Optometry, Continuing Education, The State University of New York College of Optometry, May 1, 2014, USA.
²⁴ CLARIANT, FDA Docket No. 2004N-0050, Safety and effectiveness information for piroctone olamine, submitted pursuant to 21 C.F.R. §330.14(f) and §330.10(a)(2), Aug. 13, 2004, at p. 3, USA.
²⁵ CLARIANT, Functional Chemicals Division, Taking care of your customers' hair. Date unstated. See, FIGS. 5, 6, and 7, USA/Switzerland.
²⁶ SCCNFP, Concerning piroctone olamine and its monoethanolamine salt, Opinion of the Scientific Committee on Cosmetic Products and Non-Food Products Intended for Consumers, Colipa no P59, SCCNFP/0525/01, adopted by the SCCNFP during its 19th plenary meeting of 27 Feb. 2002, see p. 2, European Union.
²⁷ SCCS, [2^{nd}] Addendum to the opinion on climbazole, Scientific Committee on Consumer Safety, Addendum SCCS/1506/13, 26 Feb. 2013; and, [1^{st}] Addendum to the opinion on climbazole, Scientific Committee on Consumer Safety, Cosmetics Europe: P64, 26 Feb. 2013, European Union; and SCCP, [Original Approval] Opinion on climbazole, Scientific Committee on Consumer Products, COLIPA n° P64, SCCP/1204/08, 21 Jan. 2009, European Union.
²⁸ RASTEGAR, et al., A comparison of clinical efficacy between a basic shampoo with herbal extracts containing climbazole and its similar sample containing piroctone olamine in the treatment of dandruff and seborrheic dermatitis, Iranian Journal of Dermatology, Vol. 12, No. 3, Autumn 2009, 82-85, Iran.
²⁹ GRICE et al., The skin microbiome, Nat. Rev. Microbiol., 2011 April; 9(4): 244-253, USA. doi:10.1038/nrmicro-2537.
³⁰ WILDE, A Study of the fatty acid metabolism of the yeast Pityrosporum ovale, Biochem. J. (1968) 108, 225-231, United Kingdom.
³¹ HOESCHT AG, Brochure, Octopirox, Hoechst AG, FDA filing, 1994, dockets/dailys/04/oct-04/101904/04n-0050-rpt-0001-E-03-Hoechst-vol-7, USA/Switzerland.
³² GAO, In vitro and in vivo killing of ocular Demodex by tea tree oil, Br. J. Ophthalmol., 2005; 89:1468-1473, United Kingdom. doi:10.1136/bjo.2005.072363.
33 DUBINI, In vitro antimycotic activity and nail permeation models of a piroctone olamine (Octopirox) containing transungual water soluble technology, Arzneimittelforschung (Drug Research) 2005, 55(8), 478-483, Germany/USA. doi: 10.1055/s-0031-1296892.
³⁴ FRANGI, In vitro antimycotic activity and biological assay of nail permeation of a new climbazole/octopirox hydrolacquer, Journal of Plastic Dermatology, September-December 2005; vol. 1, n.3, 15-19, Italy.

I claim:

1. A composition for the topical treatment or prevention of signs or symptoms associated with demodicosis, said composition comprising an effective amount of piroctone olamine.

2. The composition for the topical treatment or prevention of signs or symptoms associated with demodicosis of claim 1, wherein the effective amount of piroctone olamine comprises an amount between 0.01% and 0.49% by weight.

3. The composition for the topical treatment or prevention of signs or symptoms associated with demodicosis of claim 1, wherein said effective amount of piroctone olamine is combined with an oil and emulsified into an aqueous solution.

4. The composition for the topical treatment or prevention of signs or symptoms associated with demodicosis of claim 3, wherein said oil comprises a medium or short chain triglyceride with a carbon chain equal to or less than 10.

5. The composition for the topical treatment or prevention of signs or symptoms associated with demodicosis of claim 3, wherein said oil comprises a medium or short chain triglyceride with a carbon chain equal to or less than 8.

6. The composition for the topical treatment or prevention of signs or symptoms associated with demodicosis of claim 3, wherein said oil is comprises at least one of caprylic/capric triglyceride and fractionated coconut oil.

7. The composition for the topical treatment or prevention of signs or symptoms associated with demodicosis of claim 3, wherein said oil is emulsified with at least one of the following, a nonionic, amphoteric and zwitterionic surfactant.

8. The composition for the topical treatment or prevention of signs or symptoms associated with demodicosis of claim 1, wherein said effective amount of piroctone olamine comprises an amount of 0.10 to 0.18% piroctone olamine by weight.

9. The composition for the topical treatment or prevention of signs or symptoms associated with demodicosis of claim 1, wherein said signs or symptoms include display of signs or symptoms associated with at least one of the following: blepharitis, meibomian gland dysfunction, type 1 flushing rosacea, type 2 acne rosacea, type 4 ocular rosacea, demodicosis gravis, granulomatous rosacea, and pityriasis folliculorum.

10. The composition for the topical treatment or prevention of signs or symptoms associated with demodicosis of claim 1, comprising a topical application for treatment of a mammal displaying signs of demodectic mange.

11. The composition for the topical treatment or prevention of signs or symptoms associated with demodicosis of claim 1, further comprising an effective amount of climbazole.

12. The composition for the topical treatment or prevention of signs or symptoms associated with demodicosis of claim 11, said effective amount between 0.01% and 0.49% by weight.

13. The composition for the topical treatment or prevention of signs or symptoms associated with demodicosis of claim 12, wherein said effective amount of climbazole comprises an amount of 0.03-0.11% by weight.

14. The composition for the topical treatment or prevention of signs or symptoms associated with demodicosis of claim 11, wherein said effective amount of piroctone olamine is approximately twice the effective amount of climbazole.

15. A method for treatment of a patient exhibiting signs or symptoms associated with at least one of the following: demodicosis, blepharitis, meibomian gland dysfunction, type 1 flushing rosacea, type 2 acne rosacea, type 4 ocular rosacea, demodicosis gravis, granulomatous rosacea, pityriasis folliculorum, and demodectic mange, comprising the steps of:
a. treating mites of the genus Demodex; and,
b. treating fungi of the genus Malassezia.

16. The method for treatment of a patient of claim 15, wherein said steps of treating comprise topical application of a solution, comprising the sub-steps of:
a. wetting the patient's hair and skin with water;
b. applying an effective amount of a solution to the hair and skin;
c. lathering the solution on the hair and skin; and
d. rinsing the solution from said hair and skin using water.

17. The method for treatment of a patient of claim 16, wherein the solution comprises piroctone olamine.

18. The method for treatment of a patient of claim 17, wherein the solution additionally comprises climbazole and the ratio of climbazole to piroctone olamine is approximately 1:2.

19. The method for treatment of a patient of claim 16, further comprising repeated application daily.

20. The method for treatment of a patient of claim 16, further comprising a longer interval than daily application, and application occurs each time after the patient's hair and skin are wetted.

21. The method for treatment of a patient of claim 15, wherein said steps of treating mites and fungi comprise the sub-steps of:
a. drying the patient if wet; and
b. applying an effective amount of a topical solution to the skin of the patient.

22. The method for treatment of a patient of claim 21, wherein the topical solution comprises piroctone olamine.

23. The method for treatment of a patient of claim 22, wherein the topical solution comprises climbazole.

24. The method for treatment of a patient of claim 23, wherein the ratio of climbazole to piroctone olamine is approximately 1:2.

25. The method for treatment of a patient of claim 21, further comprising repeated application daily.

26. The method for treatment of a patient of claim 21, further comprising a longer interval than daily application, and application occurs after each time the patient's hair and skin are wetted.

27. A method for the preparation of an aqueous solution of medicinal treatments, said preparation comprising the steps of:
a. preparing a predetermined amount of dry ingredients of piroctone olamine and climbazole;
b. dissolving said dry ingredients in an alcohol;
c. dissolving said alcohol solution in oil at sufficient temperature to create an oil admixture;
d. introducing an emulsifying agent to the oil admixture to create an emulsified solution; and,
e. compounding the emulsified solution.

28. The method for the preparation of an aqueous solution of claim 27, wherein said alcohol solution is heated to approximately 170 degree F.

29. The method for the preparation of an aqueous solution of claim 28, wherein said oil is heated to approximately 170 degrees F. prior to the step of dissolving said alcohol solution.

30. The method for the preparation of an aqueous solution of claim 27, wherein said step of compounding comprises the steps of:
a. introducing said emulsified solution into water heated to approximately 170 degrees F. to create a primary solution; and
b. blending said primary solution with at least one of a hydro-colloid or hydro-gel.

31. The method for the preparation of an aqueous solution of claim 30 further comprising the steps of:
a. cooling the oil admixture; and
b. adding a preservative at a predetermined temperature.
